(19) United States
(12) Patent Application Publication
(10) Pub. No.: US 2021/0190749 A1
(43) Pub. Date: Jun. 24, 2021

(54) MACHINE OLFACTION SYSTEM AND METHOD

(71) Applicant: X Development LLC, Mountain View, CA (US)

(72) Inventors: Bennet Joseph Cobley, Mountain View, CA (US); Rohinton Mehta, Fremont, CA (US)

(21) Appl. No.: 17/028,245

(22) Filed: Sep. 22, 2020

Related U.S. Application Data
(60) Provisional application No. 62/950,617, filed on Dec. 19, 2019.

(51) Int. Cl. G01N 33/00; G01N 27/12

(52) U.S. Cl. CPC G01N 33/0034; G01N 33/0006; G01N 27/12

(57) ABSTRACT

Methods, systems, and apparatus for a multi-modal gas sensing system including a set of multi-modal gas sensor modules, where each module includes multiple gas sensors that are sensitive to a respective set of analytes. The set of modules are provided to a receptacle configured to receive modules including a gas manifold in fluidic contact with each of the modules, and a data processing apparatus in data communication with each module received by the receptacle. A sampling sequence is configured for the set of modules based in part on a set of analytes of interest representative of a test environment. A test gas is provided in turn to a first proper subset and a second proper subset of the modules and a first set and a second set of response data are collected, where the first and second sets of response data are provided as training data to a machine-learned model.

### CROSS-REFERENCE TO RELATED APPLICATIONS

This application claims the benefit under 35 U.S.C. § 119(e) of U.S. Patent Application No. 62/950,617, entitled “MACHINE OLFACTION SYSTEM AND METHOD,” filed Dec. 19, 2019. The disclosure of the foregoing application is incorporated herein by reference in its entirety for all purposes.

### BACKGROUND

Gas sensor arrays can be used to detect the presence of analytes in ambient environments surrounding the gas sensors. Detecting particular analytes in an ambient environment, e.g., volatile organic compounds, can be useful for safety, manufacturing, and/or environmental monitoring applications. Individual gas sensors can be differently sensitized to a particular subset of analytes and nonreactive to other analytes.

### SUMMARY

This specification describes systems, methods, devices, and other techniques relating to modular platform/packaged sensor assemblies to generate customizable application-specific machine olfaction systems. The modular platform/packaged sensor assemblies of differently-sensitized gas sensors can be used to generate a recognizable pattern of output signals unique to a variety of analyte compositions to which the application-specific machine olfaction systems are exposed.

In general, one innovative aspect of the subject matter described in this specification can be embodied in methods for a multi-modal gas sensing system including selecting, from multiple multi-modal gas sensor modules, a set of multi-modal gas sensor modules, where each multi-modal gas sensor module includes multiple gas sensors, and where each of the multi-modal gas sensor modules is sensitive to a respective set of analytes. The set of multi-modal gas sensor modules are provided to a receptacle configured to receive the multiple multi-modal gas sensing modules and includes a gas manifold in fluidic contact with each of the set of multi-modal gas sensor modules, and where each of the set of multi-modal gas sensor modules received by the receptacle is in data communication with a data processing apparatus. A sampling sequence is configured for the set of multi-modal gas sensor modules based in part on multiple analytes of interest representative of a test environment including determining for each analyte of interest of the multiple analytes of interest, one or more of the set of multi-modal gas sensor modules that is sensitive to the analyte of interest, selecting a first proper subset of the set of multi-modal gas sensor modules to receive a test gas including the analytes of interest based in part on a sensitivity of each multi-modal gas sensor module in the proper subset to the analytes, and selecting a second proper subset of the set of multi-modal gas sensor modules to receive the test gas comprising the analytes of interest based in part on a sensitivity of each multi-modal gas sensor module in the proper subset to the analytes. The test gas is provided to the first proper subset of multi-modal gas sensor modules and a first set of response data is collected, and the test gas is provided to the second proper subset of multi-modal gas sensor modules and subsequent to the collection of the first set of response data and a second set of response data is collected. The first set of response data and the second set of response data are provided as training data to a machine-learned model.

These and other embodiments can each optionally include one or more of the following features. In some implementations, the first proper subset of multi-modal gas sensor modules is sensitive to a first set of analytes of interest of the multiple analytes of interest, and the second proper subset of multi-modal gas sensor modules is sensitive to a second, different set of analytes of interest of the multiple analytes of interest.

In some implementations, the methods further include exposing, after providing the test gas to the first proper subset of multi-modal gas sensor modules, the first proper subset of multi-modal gas sensor modules to an inert gas for a period of time. The period of time can be an amount of time for each of the multiple sensors in the multi-modal gas sensor modules to reach a baseline resistivity reading.

In some implementations, the response data from the first proper subset and the second proper subset of multi-modal gas sensor modules includes a time-dependent response of each of the multiple gas sensors of each of the multi-modal gas sensor modules in the first proper subset and the second proper subset to the test gas. The response data from the first proper subset of multi-modal gas sensor modules can be characteristic of a first analyte of interest of the multiple analytes of interest, and where the response data from the second proper subset of multi-modal gas sensor modules is characteristic of a second, different analyte of interest of the multiple analytes of interest.

In general, another innovative aspect of the subject matter described in this specification can be embodied in a multi-modal gas sensing system including multiple multi-modal gas sensor modules, where each multi-modal gas sensor module includes multiple gas sensors including a first type of gas sensor and a second type of gas sensor different from the first type of gas sensor, such that each of the first type of gas sensor and second type of gas sensor is sensitive to a respective set of analytes. The modules further each include a housing configured to hold the multiple gas sensors, a gas inlet coupled to the housing and configured to expose the plurality of gas sensors to a gas introduced from the test environment via the gas inlet, a power supply configured to provide power to the multiple gas sensors, and circuitry. The system includes a receptacle configured to receive the multiple multi-modal gas sensing modules, and a data processing apparatus in data communication with the multiple gas sensing modules and configured to perform the operations including receiving, by the data processing apparatus and for a test environment, test instructions to test for multiple analytes of interest representative of the test environment, determining, by the data processing apparatus and using a machine-learned model trained for the test environment, a sampling sequence to detect the multiple analytes of interest, where determining the sampling sequence to detect the multiple analytes of interest includes: for each analyte of interest of the multiple analytes of interest: selecting, a subset of multi-modal gas sensor modules of the multiple multi-modal gas sensor modules that is sensitive to the analyte of interest, and collecting, by the data processing apparatus, response data for each of the multiple analytes of interest from the respective multi-modal gas sensor modules according to the sampling sequence.

These and other embodiments can each optionally include one or more of the following features. In some implementations, the system further includes a gas manifold in fluidic contact with the multiple multi-modal gas sensing modules and configured to direct the gas to the multi-modal gas sensing modules. The gas manifold can be configured to direct the gas to a subset of multi-modal gas sensing modules of the multiple multi-modal gas sensing modules at a particular time.

In some implementations, the receptacle includes circuitry to form i) module-to-module and ii) module-to-platform integration connections between the multiple multi-modal gas sensing modules and the data processing apparatus. The circuitry can include sensor interrogation circuitry, sensor tuning circuitry, and communication circuitry.

In some implementations, the multi-modal gas sensing modules include a first multi-modal gas sensing module sensitive to a first set of analytes, and a second multi-modal gas sensing module sensitive to a second, different set of analytes. The first multi-modal gas sensing module and the second multi-modal gas sensing module can be configured to receive the gas introduced from the test environment via respective gas inlets in parallel.

In some implementations, the multi-modal gas sensing modules include two or more of a same multi-modal gas sensing module sensitive to a same set of analytes. The two or more of the same multi-modal gas sensing module can be configured to receive the gas introduced from the test environment via respective gas inlets in series, and where the data processing apparatus collects response data responsive to the gas from the test environment from only a subset of gas sensors of the multiple of gas sensors of one or more respective multi-modal gas sensing modules.

Particular embodiments of the subject matter described in this specification can be implemented so as to realize one or more of the following advantages. An advantage of this technology is that off-the shelf gas sensing arrays manufactured for generic applications, e.g., industrial, agriculture, food production, can be combined into a fully customized system, where the system includes both custom hardware having a selected set of modules, and custom software for the specific application. Customized module assemblies can be generated having increased sampling frequencies and broader sensitivity to a range of analytes for the particular application. System dimensions and power draw can additionally be tailored using this approach.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example system for training an e-nose gas sensing apparatus.

FIG. 2 is a schematic of an example e-nose gas sensing module.

FIGS. 3A-3C are schematics of example configurations of the modular platform/packaged sensor assemblies.

FIG. 4 is a block diagram of an example system for training a e-nose gas sensing system including modular platform/packaged sensor assemblies.

FIG. 5 is an example operating environment for an e-nose gas sensing system including modular platform/packaged sensor assemblies.

FIG. 6 is a flow diagram of an example process of the e-nose gas sensing system.

FIG. 7 is a flow diagram of another example process of the e-nose gas sensing system.

FIG. 8 is a block diagram of an example computer system.

Like reference numbers and designations in the various drawings indicate like elements.

### DETAILED DESCRIPTION

### Overview

The technology of this patent application utilizes modular platform/packaged sensor assemblies to generate customizable application-specific machine olfaction systems.

More particularly, the technology customizes application-specific machine olfaction systems using multiple standardized multi-modal gas sensing modules, wherein each of the modules is pre-packaged to include a respective set of multiple gas sensors, sensor interrogation circuitry, sensor tuning circuitry, power management, battery power storage, communication circuitry, and module-to-module/module-to-platform integration connectors. The packaged modules can be “plug-n-play” into a receiving sub-system, where the sub-system includes circuitry to facilitate a single-stream output from the multiple modules to a data processing apparatus. Each module can include a different array of multiple gas sensors than each other array of multiple gas sensors, where each array can be sensitive to a unique set of analytes. In some embodiments, multiple identical modules that are sensitive to the same particular set of analytes can be included in the system.

Customized operation of the system can include determining a proper subset of gas sensors for each module from which to collect response data for a particular application/test environment. Moreover, operation of the modules in parallel vs series, sampling frequency, and other sampling parameters can be selected based in part on available modules in the system and application-specific needs. For example, a particular module may include gas sensors with long recovery times. In order to increase sampling frequency of the system, multiple sets of the same module are operated in sequence such that one or more of the particular module is recovering while a different instance of the particular module is being exposed to a test gas. In another example, multiple different modules may be required for a particular application, e.g., a fruit sensitized module and a pesticide sensitized module, where the different modules are each sensitive to a different set of analytes and where the different modules are operated in parallel.

The machine olfaction system can include a gas manifold to direct an unknown test gas to a particular module of the system. A particular module of the system can be selected to sniff the unknown test gas based on a recovery time of one or more of the modules in the system. The particular module of the system can also be selected to sniff the unknown test gas based on a requested information from a user, e.g., user might be interested in sniffing a particular set of analytes.

The customized application-specific machine olfaction system can be trained on-site using a cloud-based system, where the response data collected by each module including a multi-modal gas sensor array can be labeled and provided over a network to a cloud-based server to train an application and system-specific machine learned model.

### E-Nose Multi-Modal Gas Sensing Module

The e-nose multi-modal gas sensing system utilizes multiple standardized multi-modal gas sensing modules. Each of the multiple multi-modal gas sensing modules, in turn, can include a different array of multiple gas sensors than each other array of multiple gas sensors, where each array can be sensitive to a unique set of analytes. A controlled training environment can be utilized to train each of the e-nose gas sensing modules, e.g., to generate a machine-learned model for detection of multiple analytes of interest.

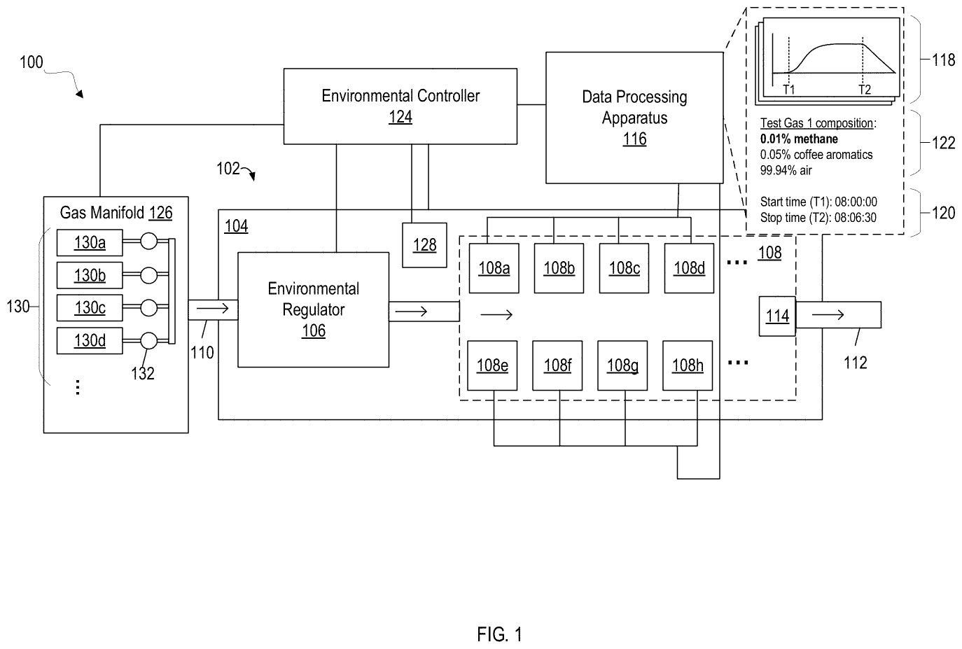

FIG. 1 is a block diagram of an example system 100 for training an e-nose gas sensing module 102. The system 100 for training an e-nose gas sensing module 102 can include a controlled environment, e.g., a laboratory setting, where external environmental factors, e.g., temperature, humidity, presence of chemicals/gases, is highly controlled and/or regulated.

The gas sensing module 102 includes a housing 104 including an environmental regulator 106. Environmental regulator 106 can include a heat-exchange component, e.g., cylindrical heaters inserted into the housing, and/or heat transfer fins for controlling the temperature of gases that are introduced through the gas inlet 110. The heat-exchange component can be configured to interact with the gas and regulate a temperature of the gas to the particular temperature and prior to entering the gas inlet.

Environmental regulator 106 can be configured to control a temperature within the housing 104, gas sensors 108, and a gas within the housing 104, for example, to a temperature between 40-45° C., to a temperature above dew point >16° C., or at a temperature relevant to an environment of interest (e.g., room temperature 23° C.). In some implementations, environmental regulator 106 can be configured to regulate a relative humidity within the housing 104, gas sensors 108, and a gas within the housing 104, e.g., to a relative humidity below 10%, to a relative humidity between 10-30%, or to a relative humidity relevant to an environment of interest.

The housing 104 and environmental regulator 106 can be in thermal contact such that the gas introduced through the gas inlet 110, the gas sensors 108, and the housing 104 are all maintained at a same temperature during operation of the module 102.

Housing 104 can be composed of various materials that are selected to be nonreactive to a set of analytes to which the housing 104 will be exposed. Materials for the housing 104 can include, for example, Teflon, Teflon-coated aluminum or stainless steel, Delrin, or other materials that are resistant to the set of analytes.

Housing 104 includes fixtures to hold a set of gas sensors 108 within the housing 104. The fixtures can be configured to accommodate particular dimensions of the gas sensors, and a layout of the fixtures within the housing 104 can be configured to designate particular locations for different types of gas sensors 108 within the housing 104.

Housing 104 further includes a gas inlet 110 and a gas outlet 112, where the gas inlet 110 is configured to allow for the introduction of gases into the housing 104 and to flow gas across the gas sensors 108. Gas outlet 112 is configured to allow for the purge of the gases from the housing 104.

Gas inlet 110 and gas outlet 112 can be configured for gas flow rates ranging between 0-10 cubic feet/hour, e.g., 5 cubic feet/hour. A particular flow rate for a gas into the gas inlet 110 can be selected, for example, based on an amount of time it takes for the environmental regulator 106 to bring a gas introduced at the gas inlet 110 to a test temperature, e.g., how long the gas will have to be in the fins of the environmental regulator 106 to get it to the temperature of the housing 104.

In some implementations, gas sensing module 102 includes a fan 114 configured to generate a negative pressure at the gas inlet 110 and within the housing 104 which can draw a gas into the housing 104 via the gas inlet 110, move the gas across the gas sensors 108, and purge the gas from the gas outlet 112. One or more operating parameters of the fan 114, e.g., a rotational speed of the fan, can be selected to regulate a desired flow rate of the gas through the gas sensing module 102.

Gas sensors 108 include a multi-modal array of gas sensors that can be sensitive to various different organic and/or inorganic compounds. In other words, the multi-modal array of gas sensors 108 can include gas sensors that are responsive to certain analytes in a test gas and not sensitive to others. Types of gas sensors 108 can include gas sensors having different sensing mechanisms, e.g., metal oxide (MOx) sensors, photoionization detector (PID) sensors, electrochemical sensors, nondispersive infrared (NDIR) sensors, or other types of gas sensors. For example, gas sensors included in a gas sensing module 102 include MOx sensors 108a-c, PID sensors 108d-f, electrochemical sensor 108g, and NDIR sensor 108h.

In some implementations, types of gas sensors 108 include gas sensors having a same sensing mechanism, e.g., oxidation-based, resistivity-based, optical-based, etc., but can have different sensitivities to the multiple analytes. In other words, a first type of gas sensor 108a and a second type of gas sensor 108b have a same mechanism for gas sensing, e.g., MOx sensors that make resistivity-based measurements, but are configured to have different performance parameters, e.g., a MOx sensor operating at a first voltage bias and a MOx sensor operating at a second voltage bias, such that the respective sensitivities to certain analytes are different.

The multi-modal array of gas sensors 108 can include multiple gas sensors, e.g., 38 total gas sensors, 25 total gas sensors, greater than 40 total gas sensors. A number of each type of gas sensor relative to respective numbers of each other type of gas sensor included in the multi-modal array of gas sensors 108 can depend in part on dimensional/size considerations, cost-benefit of each type of sensor, responsivity, signal-to-noise ratio of each sensor, or the like. A field of application of the sensor array, e.g., agricultural, industrial, etc., can determine a number of each type of gas sensor relative to a respective number of each other type of gas sensor in the multi-modal array of gas sensors 108. For example, for applications that may have a fainter signal-to-noise ratio, e.g., more background analytes not of interest, more sensors will be included overall in the multi-modal array of gas sensors 108.

In some implementations, multiple different gas sensors can each have a same sensitivity to a particular analyte, e.g., a first type of gas sensor and a second type of gas sensor can each be sensitive to the particular analyte.

The array of gas sensors 108 can be configured within the house 104 such that a test gas introduced via an inlet 110 can be sampled simultaneously by all of the gas sensors 108 in the array of gas sensors. In other words, the test gas is exposed to the multiple gas sensors in the array of gas sensors simultaneously such that data collection from each of the multiple gas sensors 108 can be performed in parallel.

In some implementations, the multi-modal array of gas sensors can include multiple MOx sensors 108a-c that are each configured to operate at a different temperature, e.g., biased at a different operating voltage, such that they have different responses to particular analytes based on the operating parameters.

Each gas sensor 108 is connected to a data processing apparatus 116 which is configured to collect data from the gas sensors 108, e.g., response data 118. Response data 118 can include a time-dependent response of each of the gas sensors 108 to a test gas. Additionally, the data processing apparatus 116 can be configured to collect time-dependent measurements of operating conditions of the plurality of sensors 108, gas inlet 110, and environmental controller 124, e.g., temperature and relative humidity, gas flow rate, and the like. In some implementations, response data 118 includes a measure of the resistivity of the gas sensor 108 versus time.

Data processing apparatus 116 can be hosted on a server or multiple servers in data communication with the gas sensors over a network. The network may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. The network may include multiple networks or subnetworks, each of which may include, for example, a wired or wireless data pathway. The network may include a circuit-switched network, a packet-switched data network, or any other network able to carry electronic communications (e.g., data or voice communications). For example, the network may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. The network may include one or more networks that include wireless data channels and wireless voice channels. The network may be a wireless network, a broadband network, or a combination of networks including a wireless network and a broadband network.

Data processing apparatus 116 can be configured annotate the response data 118 received from the gas sensors 108 responsive to a test gas. In some implementations, annotation data 120 includes timestamps, e.g., a start time label, a stop time label, and/or respective composition data 122 of the test gases, e.g., a set of known analytes of interest and set of known analytes not of interest, being evaluated using the gas sensing module 102.

In some implementations, the data processing apparatus 116 is configured to generate annotation data 120 before, during, and after exposure of a test gas to the gas sensors 108, where the annotation data 120 includes recording a first label describing a first state of the multi-modal gas sensing array, e.g., a start time of the gas exposure. The gas manifold 126 can provide a first test gas to the multi-modal array of gas sensors 108, where the first test gas includes a first concentration of a known analyte of interest and a second concentration of a known analyte that is not of interest, e.g., a confounding gas. The data processing apparatus 116 can then collect a set of response data 118 from each of the gas sensors 108 of the multi-modal gas sensing array responsive to the first test gas and then record annotation data 120, e.g., a second label, describing a second state of the multi-modal gas sensing array, e.g., a stop time of gas exposure.

In some implementations, the data processing apparatus 116 further generates, from the response data 118, the annotation data 120, and the composition data 122 for the known test gases, training data for a machine-learned model for the module 102.

In some implementations, the data processing apparatus 116 is in data communication with an environmental controller 124. The environmental controller 124 may be a part of the data processing apparatus 116. The environmental controller 124 can be configured to provide operating instructions to the environmental regulator 106, gas sensors 108, data processing apparatus 116, and gas manifold 126. In some implementations, the environmental controller 124 can be configured to receive operational feedback, e.g., solenoid valve status, temperature control, etc., from one or more of the environmental regulator 106, gas sensors 108, data processing apparatus 116, and gas manifold 126.

In some implementations, the environmental controller 124 can receive operating conditions feedback, e.g., temperature, humidity readings, from a temperature and/or humidity gauge 128. The gauge 128, e.g., a thermocouple, hygrometer, or the like, can be in physical contact with the housing 104 or gas sensors 108 to measure a temperature and/or relative humidity. The gauge 128 can additionally or alternatively measure a temperature and/or relative humidity of the gas present within the housing 104.

In some implementations, the operating conditions feedback received by the environmental controller 124 can be provided to the data processing apparatus 116 and recorded as annotation data 120. For example, temperature of the housing 104, gas sensors 108, and test gas can be recorded as annotation data 120 and included with the response data 118 for the particular test gas.

The system 100 further includes a gas manifold 126 having multiple gas sources 130. The multiple gas sources 130 can each include multiple known analytes of interest and multiple known analytes not of interest. For example, gas manifold 126 can include gas source 130a, 130b each having analytes of interest and gas sources 130c and 130d each having analytes not of interest (e.g., confounding gases).

Gas manifold 126 can be a closed-loop system, where no external compounds are present within the gas manifold 126 that could interfere with the test gases provided by the gas manifold to the gas sensing module 102 via the gas inlet 110. The test gases provided by the gas manifold 126 to the gas sensing module 102 can be composed only of the known analytes of interest and known analytes not of interest from the one or more gas sources 130.

Gas manifold 126 can further include regulatory components 132 which can be operable to selectively allow a controlled flow (e.g., 0-5 cubic feet/hour) of one or more of the gas sources 130 from the gas manifold 126 and into the gas inlet 110 to generate a particular test gas for the gas sensing module 102.

For a particular test gas provided by the gas manifold through the gas inlet 110 of the e-nose gas sensing module 102, response data 118 is collected from each of the gas sensors 108 of the gas sensing array. The gas sensors 108 can be a multi-modal array of gas sensors having a variety of response characteristics to a range of analytes. Different types of gas sensors 108 can be more or less responsive to a particular analyte, and a subset of the gas sensors 108 can be identified as optimally responsive to the particular analyte based in part on the response data 118 collected.

In some implementations, response of a gas sensor 108 to a test gas including one or more analytes can be measured as a change in gas sensor electrical resistivity before, during, and after exposure to the particular test gas (e.g., as in MOx sensors). In some implementations, response of a gas sensor 108 can be measured as an electrical signal generated by one or more analytes of interest that are present in the test gas (e.g., as in PID sensors).

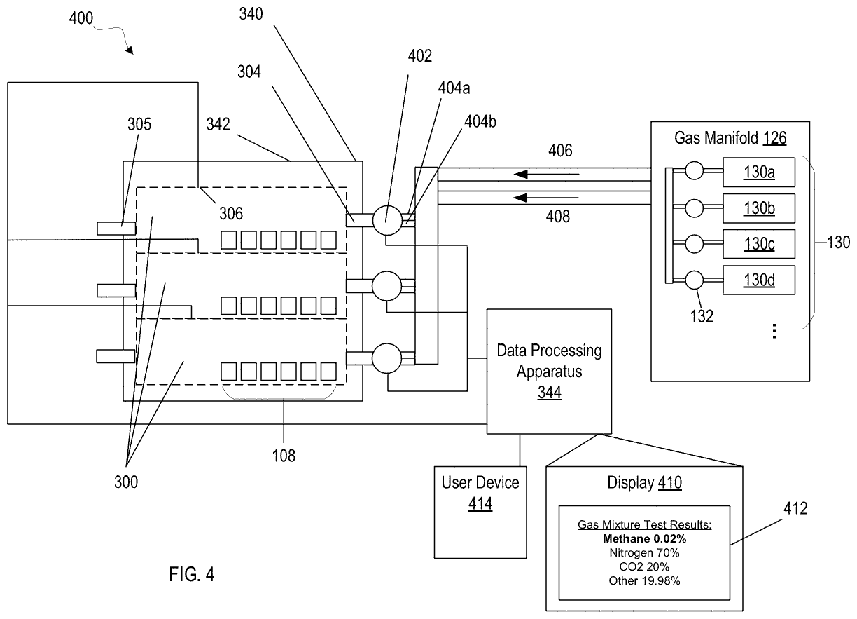

As described with reference to FIG. 1, a particular gas sensing module 102 is exposed to test gases provided by the gas manifold 126 and response data 118 is collected from the gas sensors 108 to generate training data for a machine-learned model. Multiple gas sensing modules 102 included in a gas sensing system can similarly be exposed to test gases provided by a gas manifold 126 to generate response data 118 and generate training data for a machine-learned model, as described in further detail with reference to FIGS. 4-7 below.

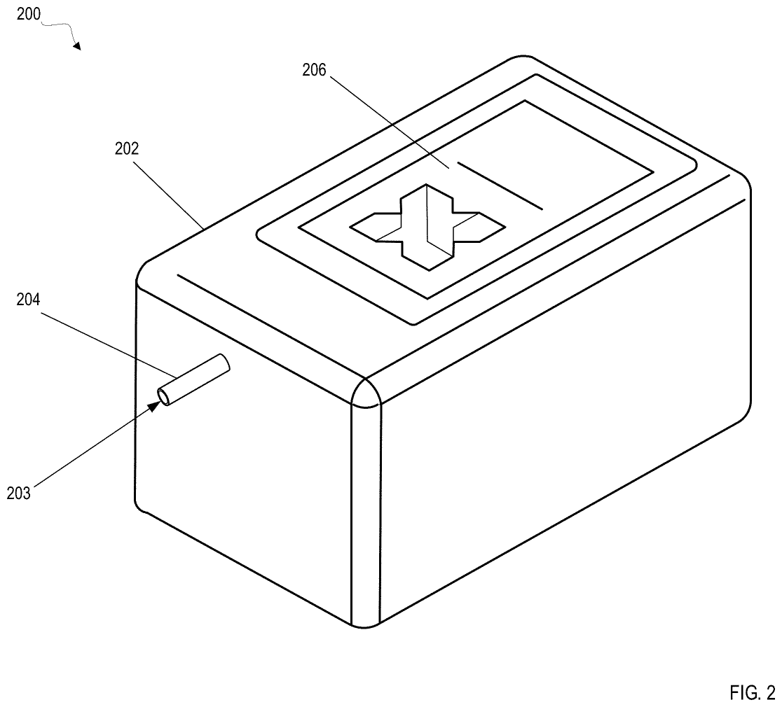

FIG. 2 depicts an example exterior of an e-nose gas sensing module 200, e.g., gas sensing module 102. The gas sensing module 200 can include housing 202, e.g., housing 104, enclosing the various components of the gas sensing module 200. A gas inlet 204, e.g., gas inlet 110, can receive an unknown test gas 203, and provide the unknown test gas to a multi-modal gas sensing array, e.g., a multi-modal array of gas sensors 108, within the housing 202. Gas sensing module 200 can include a display 206, which can include a touchscreen. Display 206 can provide an intuitive user interface for receiving user instructions, e.g., run test, testing parameters, etc., and display information to a user viewing the display 206.

A footprint, e.g., width and length, of the gas sensing module 200 can be, for example, smaller than 2×4 inches, smaller than 10×12 inches, smaller than 20×20 inches, or the like. Though depicted in FIG. 2 as having a rectangular form factor, other form factors are possible, e.g., cylindrical form factor. In one example, the gas sensing module 200 can have dimensions similar to a standard shoebox, e.g., 14×8×5 inches. In some implementations, a footprint of the gas sensing module 200 can be fit to the dimensions of a silicon chip, e.g., on the order of 1 mm×1 mm×0.1 mm or smaller.

### E-Nose Multi-Modal Gas Sensing Modular Platform System

As described above, multiple gas sensing modules 102 can be combined into a e-nose multi-modal gas sensing system, where the multiple gas sensing modules 102 can be selected to be incorporated into a multi-modal gas sensing modular platform system based in part on an application of the multi-modal gas sensing system, and where a machine-learned model can be trained based on the application-specific set of analytes of interest that are characteristic the application, e.g., using a test environment.

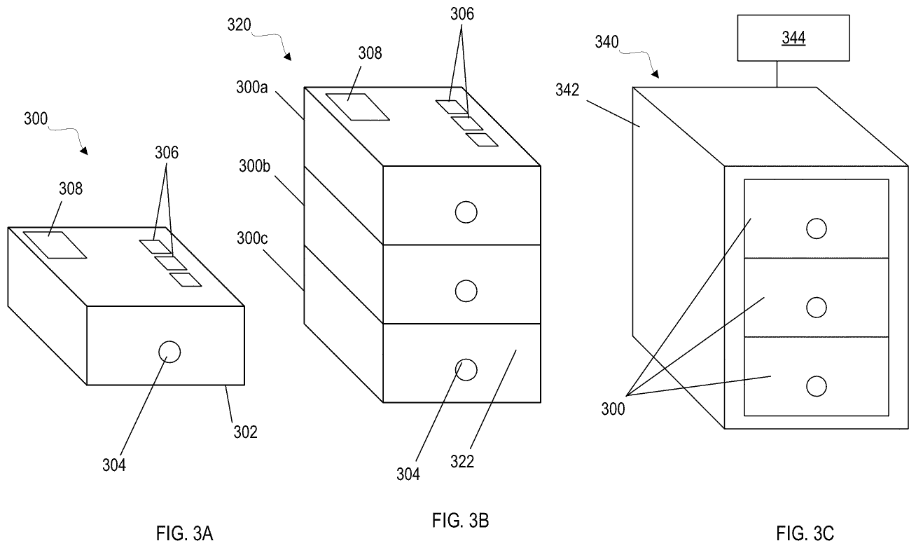

FIGS. 3A-3C are schematics of example configurations of the modular platform/packaged sensor assemblies. Referring to FIG. 3A, gas sensor module 300, e.g., gas sensor module 102, is a modular platform/packaged sensor assembly. As described with reference to FIGS. 1 and 2, each gas sensing module 300 includes a housing 302, e.g., housing 104, and gas inlet 304, e.g., gas inlet 110. Circuitry 306 can be located on the housing 302 of the gas sensor module 300, where the circuitry 306 includes a set of contacts allow for access out on an outside surface of the module 300 to the internal control circuitry, gas sensor readouts, and the like from inside the housing 302. For example, response data from the gas sensors, e.g., gas sensors 108, located within the housing 302 of the gas sensor module 300 can be accessed via the circuitry 306, e.g., by making electrical contact with the circuitry 306.

Module 300 additionally includes a power supply 308 selected to provide necessary operational power to gas sensors 108 of the module 300. The power supply 308 can additionally be selected to provide operational power to the various other components of the module 300 including the data processing apparatus 116, environmental regulator 106, controller 124, etc.

FIG. 3B is a schematic of an example stack 320 of gas sensor modules 300 for a gas sensor system. Gas sensor modules 300a, 300b, and 300c can be configured with respect to one another, e.g., stacked, such that the respective gas inlets 304 of each of the modules 300a-c are accessible at a same side 322 of the stack 320 of modules 300. A test gas can therefore be introduced to each of the respective gas inlets 304 of the modules 300 along a same side 322 of the stack 320. Circuitry 306 corresponding to each of the modules 300 of the stack 320 can be configured to be accessible when the modules 300 are arranged in the stack 320, e.g., through corresponding circuitry on a receiving receptacle for the stack 320.

FIG. 3C is a schematic of an example e-nose gas sensing system 340 including multiple modular platform/packaged sensor assemblies. A receptacle 342 is configured to receive multiple modules 300. Modules 300 can be stacked within receptacle 342, e.g., according to a stack 320, such that the circuitry 306 for the respective modules 300 is accessible by corresponding circuitry in the receptacle 342, which allows for a data processing apparatus 344, e.g., a data processing apparatus 116, for the e-nose gas sensing system 340 to access the operations of each of the modules 300 when the modules 300 are received by the receptacle 342. The circuitry in the receptacle 342, e.g., sensor interrogation circuitry, sensor tuning circuitry, and/or communication circuitry, can be configured to form module-to-module and module-to-platform integration connections between the multiple modules 300 and the data processing apparatus 344 when the modules are received by the receptacle 342.

The data processing apparatus 344 can be configured to access the operations and collect response data, e.g., response data 118, from the respective gas sensors 108 of each of the modules 300 in the e-nose gas sensing system 340. Configuration and operation of the e-nose gas sensing system 340 is described below with respect to FIGS. 4-7.

FIG. 4 is a block diagram of an example system 400 for training an e-nose gas sensing system 340 including multiple modular platform/packaged sensor assemblies 300. E-nose gas sensing system 340, includes multiple modules 300 each including multiple multi-modal gas sensors 108. The multiple multi-modal gas sensors 108 can include a first type of gas sensor, e.g., an organic-type gas sensor, and a second type of gas sensor, e.g., an inorganic-type gas sensor, that is different from the first type of gas sensor. Each of the first type and second types of gas sensors are sensitive to a respective set of analytes, e.g., a first type of gas sensor can be sensitive to volatile organic compounds (VOCs) and a second type of gas sensor can be sensitive to inorganic compounds.

The e-nose gas sensing system 340 can be in fluidic contact with a gas manifold 126 including gas sources 130, as described above with respect to FIG. 1. The different modules 300 of the e-nose gas sensing system 340 can be selectively isolated from the gas manifold 126 via a set of valves 402, e.g., solenoid valves, compressed air control valves, on/off valves, or other similar flow-control valves. Each valve 402 can be located between the gas inlets 304 of the e-nose gas sensing system and the gas manifold 126 to isolate a respective module 300 from the gas manifold 126. Multiple gas inlets 404 can be provided to the manifold side of the valve 402, each of the gas inlets 404 configured to provide a fluidic pathway for a gas from the gas manifold 126 to the valves 402.

As depicted in FIG. 4, a first gas inlet 404a can be utilized to provide a test gas 406 to the e-nose gas sensing system 340 via gas inlet 304 and a second gas inlet 404b provides a purge gas 408 to the e-nose gas sensing system 340 via gas inlet 304. Each valve 402 can include multiple input ports 404a, 404b located on a manifold side of the valve 402 and can switch between an “off” position, a first position to allow test gas 406 to the gas inlet 304, e.g., via gas inlet 404a, and a second position to allow purge gas 408 to the gas inlet 304, e.g., via gas inlet 404b.

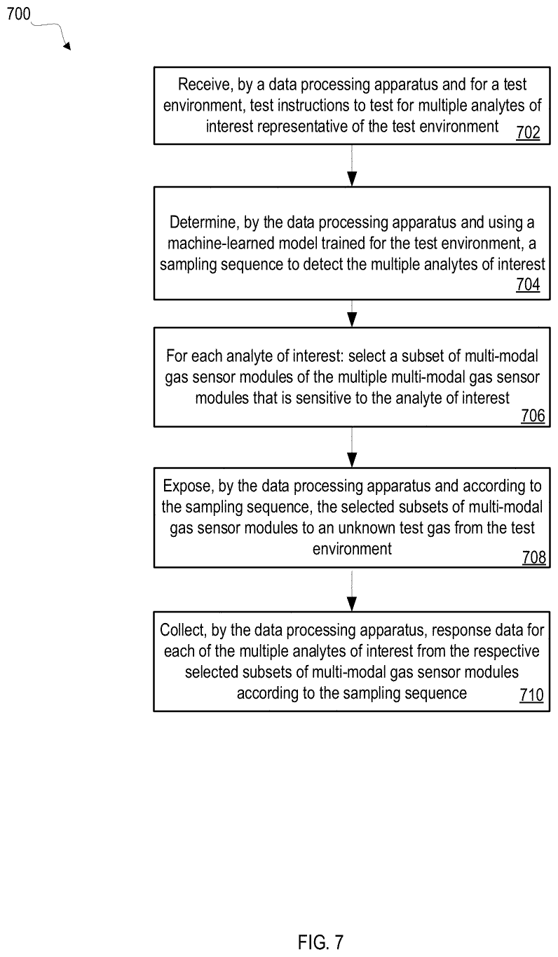

In some implementations, the operation of valves 402 can be controlled by data processing apparatus 344, e.g., to selectively allow a test gas 406 into a respective module 340, a purge gas 408 into a respective module 340, or to isolate a valve 402, e.g., based on a sampling sequence for the e-nose gas sensing system 340. Further details of generating a sampling sequence for the e-nose gas sensing system 340 are discussed with reference to FIG. 7.

E-nose gas sensing system 340 further includes gas outlets 305, e.g., gas outlet 112, to evacuate any gasses, e.g., test gas 406 or purge gas 408, from the respective modules 340 of the e-nose gas sensing system 340.

In some implementations, data processing apparatus 344 includes a display 410 to provide information 412 to a user of the gas sensing system 340 and/or allow for user interaction with the gas sensing system 340. Information 412 can include, for example, the operational status of the system 340, e.g., on/off, testing, processing, etc.

In some implementations, information 412 includes test results for the test gas 406. Information 412 can be presented to a user based on user preferences, e.g., to highlight a particular set of analytes that the user is interested in discovering in the test gas 406. Information 412 can be additionally or alternatively provided to one or more user devices 414 in data communication with the system 340.

In some implementations, display 410 is configured to respond to user interaction, e.g., a touch-screen functionality. Display 410 can further include audio feedback, e.g., an alert, to notify a user of the status of the system 340. For example, the system 340 can provide an audio and/or visual update to the user of a testing status. In another example, the system 340 can provide an audio and/or visual alarm to the user, e.g., if a particular analyte is detected above/below a preset threshold, e.g., a threshold concentration of the analyte is detected in the ambient.

In some implementations, imaging data can be captured of an environment surrounding the system 340, e.g., using a camera or video recording device (not shown). The imaging data of the surrounding environment can be displayed on display 410 to identify, to a user, one or more objects in the surrounding environment that may be included in the test gas 406 being sampled by the system 340. A blend ratio of the various analytes that are being sensed in the test gas 406 can be displayed on display 410.

In some implementations, one or more of the functions of the data processing apparatus 116 can be performed on a user device 414, where a user can interact with the gas sensing system 340 via the user device 414 e.g., receive data, provide testing instructions, receive testing information, or the like. User device 414 can include, for example, a mobile phone, tablet, computer, or another device including an application environment through which a user can interact with the gas sensing system 340. In one example, user device 414 is a mobile phone including an application environment configured to display test gas test results for the test gas 406, allow for user interaction with the gas sensing system 340, and the like.

The data processing apparatus 344 can be an onboard computer that is affixed to the receptacle 342 of the system 340. In some implementations, a portion or all of the data processing apparatus 344 can be hosted on a cloud-based server that is in data communication with the gas sensing system 340 over a network.

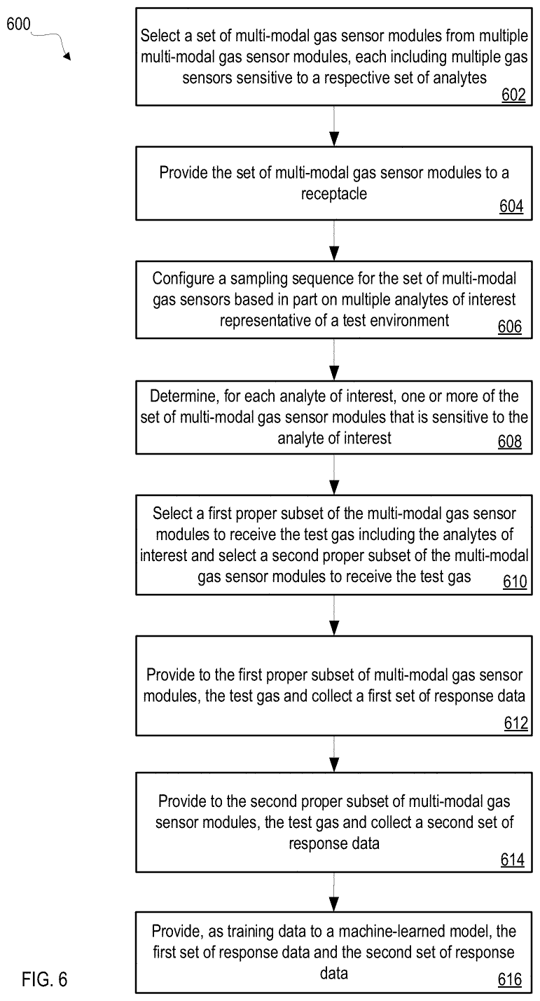

System 400 for training the e-nose gas sensing system 340 can generate training data for multiple known analytes of interest using gas sources 130 of the gas manifold 126. In an analogous method to what is described above with reference to FIG. 1 for configuring the gas sensors 108 of a particular gas sensing module 102 using system 100, each module 300 of the e-nose gas sensing system 340 may be configured for a particular set of known analytes using system 400. A method for generating training data to train a machine-learned model for the e-nose gas sensing system 340 for a particular application/environment is described with reference to FIG. 6.

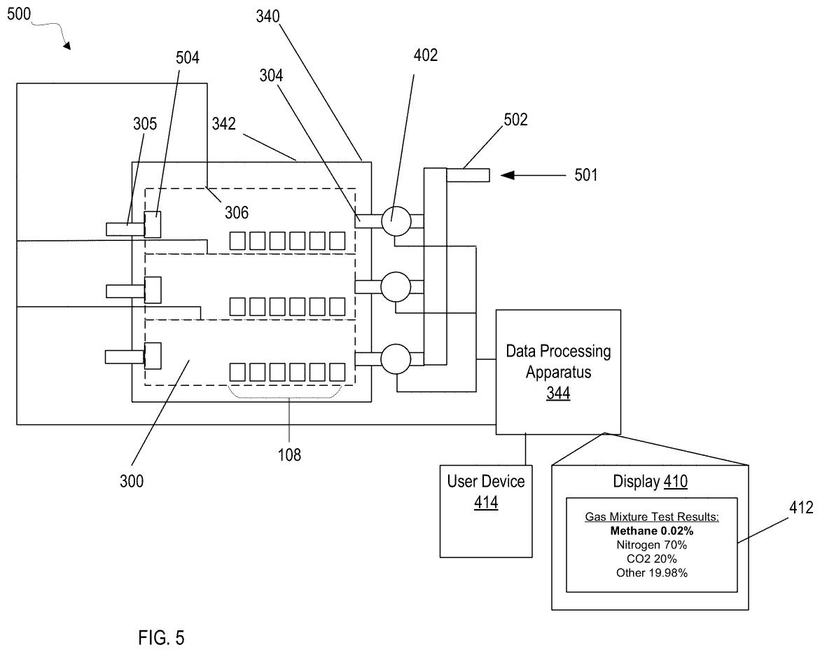

FIG. 5 is an example operating environment 500 for an e-nose gas sensing system 340 including modular platform/packaged sensor assemblies, e.g., modules 300. As discussed above with reference to FIG. 4, the e-nose gas sensing system 340 includes multiple modules 300, each including a respective gas inlet 304 configured to receive an unknown test gas 501 and a gas outlet 305 through which the unknown test gas 501 is purged from the respective module 300 of the e-nose gas sensing system 340.

Unknown test gas 501 can be introduced passively via the environment surrounding the gas inlet 502. For example, the unknown test gas 501 can be introduced via the gas inlet 502 when the gas sensing system 340 is deployed in a testing environment, e.g., in a factory setting. Passive introduction of the unknown test gas 501 into the gas inlet 502 can be, for example, by diffusion of the unknown test gas 501 into the gas inlet 502.

In some implementations, the unknown test gas 501 can be introduced actively into the gas inlet 502, for example, by generating a negative pressure within the one or more of the modules 300 using a respective fan 504, e.g., fan 114, or other similar device. In another example, the unknown test gas 501 can be introduced actively to the gas inlet 502 by a positive pressure of the gas at the gas inlet, e.g., by a person blowing into the gas inlet 502, a gas exhaust from a piece of equipment, or the like.

Data processing apparatus 344 can be configured to operate the e-nose gas sensing system 340 in a particular sampling sequence to analyze the unknown test gas 501 using the multiple available modules 300 of the system 340. Based on the determined sampling sequence, the data processing apparatus 344 can actuate one or more of the valves 402 in a particular sequence to introduce the unknown test gas 501 to the selected modules 300 according to the sampling sequence.

In some implementations, a sampling sequence for the system 340 can depend in part on a set of analytes of interest to detect in the unknown test gas 501, where one or more of the modules 300 is sensitive to one or more analytes of the set of analytes of interest. The operation of e-nose gas sensing system 340 in a test environment is described in more detail with reference to FIG. 7 below.

### Example Operation of E-Nose Multi-Modal Gas Sensing Apparatus

The e-nose multi-modal gas sensing modular platform/packaged sensor assembly system can operate in various modes, including a configuration mode, e.g., determining a subset of gas sensors to include in the gas sensing apparatus for a particular environment including a set of analytes of interest, a training mode, e.g., training a machine-learned model to identify various analytes of interest, and detection mode, e.g., where the e-nose multi-modal gas sensing apparatus is deployed in a testing environment.

Training data can be generated using system 340 and provided to train a machine-learned model which can then be deployed in a test environment to detect one or more analytes. Multiple sets of training data can be generated, where each set of training data can be customized for a particular test environment, e.g., a factory environment, an agricultural environment, a home environment, etc., such that the machine-learned model is trained to recognize a set of analytes that are relevant to the environment, e.g., process gases to a fabrication environment, and of importance to the particular environment, e.g., detecting toxic chemicals rather than inert chemicals.

A set of modules 300 can be selected to be included in an e-nose gas sensing system 340 based in part on a set of analytes of interest that are representative of a test environment into which the e-nose gas sensing system 340 will be deployed. One or more of the modules may be specifically configured to be sensitive to a particular set of analytes of interest, e.g., organic, agricultural-related analytes, or inorganic, toxic analytes. Each of the pre-configured modules 300 can be selected for inclusion into the e-nose gas sensing system 340 based on the set of analytes of interest to be measured and the respective sets of analytes to which each module 300 is sensitive.

FIG. 6 is a flow diagram of an example process 600 of the e-nose gas sensing system 340. A set of multi-modal gas sensor modules are selected from multiple multi-modal gas sensor modules, each including multiple gas sensors that are sensitive to a respective set of analytes (602). Gas sensor modules 300, e.g., gas sensor module 102, each include multiple multi-modal gas sensors 108, where the multi-modal gas sensors 108 include at least a first type of gas sensor and a second, different type of gas sensor, e.g., a MOx sensor and a PID sensor. The set of gas sensor modules 300 can include a first module with a first set of multi-modal gas sensors 108 and a second module 300 with a second, different set of multi-modal gas sensors 108. In one example, the set of gas sensor modules 300 includes a first gas sensor module 300 that is sensitive to detect VOCs associated with fruits, and a second gas sensor module 300 that is sensitive to detect pesticides associated with agriculture.

In some implementations, two modules 300 that are selected to be included in the e-nose gas sensing system 340 can include a same set of gas sensors 108 but can be configured, e.g., using different machine learned models, to be sensitive to different sets of analytes of interest. In other words, each of the modules 300 can be trained separately using the methods described with respect to FIG. 1 and then included in a system 340 to each be sensitive to their respective sets of analytes of interest.

In some implementations, each of the modules 300 of the set of multi-modal gas sensors modules 300 selected can be sensitive to a different set of analytes, e.g., a first module can be sensitive to organic compounds, and a second module can be sensitive to inorganic compounds. The set of multi-modal gas sensor modules 300 can include two or more of a same type of gas sensor module 300, in other words, duplicate modules 300 that include a same set of gas sensors 108.

The set of multi-modal gas sensor modules are provided to a receptacle (604). Receptacle 342 can include the necessary hardware, e.g., clips, plugs, or other standardized attachment points, to receive the multi-modal gas sensor modules 300 in a “plug-and-play” format, where each of the modules 300 can be inserted into the receptacle and are held fixed within the receptacle at one or more attachment points. As depicted in FIGS. 3C, 4, and 5, the gas sensor modules 300 are received by a receptacle 342, where the receptacle includes control circuitry corresponding to the circuitry 306 of each of the modules 300 to enable data communication between the modules 300 and data processing apparatus 344, e.g., provide operating instructions, collect response data 118, and the like.

The receptacle 342 can further include fluidic connections, e.g., tubing, valves, and the like, to a gas manifold 126 including multiple gas sources 130 to provide a test gas 406 and purge gas 408 to the modules 300 within the receptacle 342. As described with reference to FIGS. 4 and 5, the receptacle 342 further includes a set of valves 402 coupled to the respective gas inlets 304 of each of the modules 300, where the valves 402 can be selectively actuated between multiple positions to 1) isolate the module 300, 2) allow for a test gas 406 to be exposed to the gas sensors 108 of the module 300, or 3) allow for a purge gas 408 to be exposed to the gas sensors 108 of the module 300.

A sampling sequence for the set of multi-modal gas sensors is configured based in part on multiple analytes of interest representative of a test environment (606), where the sampling sequence can be configured in a first step by determining, for each analyte of interest of the multiple analytes of interest, one or more of the set of multi-modal gas sensor modules that is sensitive to the analyte of interest (608).

A machine-learned model for an e-nose gas sensing system 340 may be trained using training data generated using system 400, where the machine learned model is trained to detect multiple analytes of interest representative of a test environment. The sampling sequence can be selected to expose particular modules 300 of the gas sensing system 340 at a given time which are sensitive to one or more of the multiple analytes of interest. In step 608, for each analyte of interest of the multiple analytes of interest that are representative of a test environment, a subset of the modules 300 is identified that are included in the gas sensing system 340 that are sensitive to the particular analyte of interest.

The analytes of interest can be identified based on an intended environment for the e-nose gas sensing system 340 with the selected set of gas sensor modules 300. For example, an industrial setting may have an interest in solvent analytes or processing gases. In another example, for an agricultural setting, analytes of interest are methane, pesticide compounds, or other agricultural by-products. In yet another example, for a laboratory setting, an analyte of interest can be a toxic or hazardous gas, e.g., chlorine gas. The analytes not of interest, or “confounding” gases can be selected to be inert gases, e.g., nitrogen, or as gases that are typically present in the intended environment for the apparatus with the selected set of gas sensors. For example, in a laboratory environment, an analyte not of interest can be nitrogen, a solvent, e.g., isopropyl alcohol, or carbon dioxide. In another example, in an agricultural setting, analytes not of interest can be carbon dioxide, equipment exhaust, e.g., vehicle exhaust, or gasoline.

In some implementations, determining which subset of modules 300 to select includes determining which modules 300 have at least a threshold sensitivity to the particular analyte of interest. In other words, that at least one or more of the gas sensors 108 of each of the subset of modules 300 has at least a threshold sensitivity to the particular analyte of interest.

In some implementations, determining the subset of modules 300 to select for a particular analyte of interest includes determining a classification of the analyte of interest, e.g., organic vs inorganic, and comparing the classification of the analyte of interest to a classification for the type of module 300, e.g., module configured to detect organic compounds vs a module configured to detect inorganic compounds.

Referring back to FIG. 6, the sampling sequence is further configured by selecting a first subset of the multi-modal gas sensor modules to receive the test gas including the analytes of interest and selecting a second subset of the multi-modal gas sensor modules to receive the test gas including the analytes of interest (610). The sampling sequence defines an order of sampling for the gas sensing system 340 to “sniff” for multiple analytes of interest, where one or more of the multiple analytes of interest is measured by the gas sensing system at a time. A first subset of the multi-modal gas sensor modules can be fewer than a total number of modules 300 included in the gas sensing system 340, and can be selected based on the determination that the selected first subset of modules 300 is sensitive to a first set of analytes of interest of the multiple analytes of interest. A second subset of the multi-modal gas sensor modules 300 can be fewer than a total number of modules 300 included in the gas sensing system 340, and can be selected based on the determination that the selected second subset of modules 300 is sensitive to a second set of analytes of interest of the multiple analytes of interest.

The test gas is provided to the first proper subset of multi-modal gas sensor modules, and a first set of response data is collected (612). Referring to FIG. 4, the test gas 406 can be provided to the first subset of multi-modal gas sensor modules 300 by selectively allowing the flow of the test gas 406 to the selected modules 300 via respective gas inlets 304 using the valves 402, e.g., opening the valves 402 to select gas inlet 404a.

The test gas can be produced using a gas manifold, e.g., gas manifold 126, including multiple gas sources, e.g., gas sources 130. Each of the multiple gas sources 130 can include a different analyte, where the analytes can be mixed together using gas manifold 126 to generate a particular composition of known analytes in the test gas. The test gas can include a first concentration of a known analyte of interest and a second concentration of a known analyte not of interest. For example, 0.01% of methane and 0.99% of nitrogen or air.

A data processing apparatus 344 collects response data, e.g., response data 118, generated by each gas sensor module 300 of the first subset of gas sensor modules 300 in response to the test gas 406, where the generated response data 118 from each gas sensor of the multiple gas sensors 108 in each respective module 300 of the first subset of gas sensor modules includes a response of the gas sensor to one or more analytes of the multiple known analytes in the test gas 406. Data processing apparatus 344 can collect response data 118, annotation data 120, and composition data 122 for each gas sensor 108 in response to the test gas, as described with reference to FIG. 1.

In some implementations, response data includes a response of the gas sensor 108 over a period of time. In one example, response data includes a measure of a change in electrical resistivity of the gas sensor over time. Each of a first type of gas sensor and the second type of gas sensor can have a different response to the multiple known analytes of the test gas. For example, an organic-type gas sensor may react to an organic analyte, e.g., methane, in the test gas and not react to an inorganic analyte, e.g., tin oxide, in the test gas, and an inorganic-type gas sensor may not react to the organic analyte in the test gas and react to the inorganic analyte in the test gas.

A gas sensor can have no response or a response below a threshold response to a particular analyte. A gas sensor can have a threshold response to a particular analyte. In some implementations, a change in electrical resistivity of a gas sensor 108 is measured prior to exposure to the test gas, e.g., timestamp T1 in response data 118, during exposure to the test gas, and after termination of exposure to the test gas, e.g., timestamp T2 in response data 118. A plot of the response of the gas sensor versus time of collection can be recorded for each gas sensor by the data processing apparatus.

The test gas is provided to the second subset of multi-modal gas sensor modules, and a second set of response data is collected (614). Referring to FIG. 4, the test gas 406 can be provided to the second subset of multi-modal gas sensor modules 300 by selectively allowing the flow of the test gas 406 to the selected modules 300 via respective gas inlets 304 using the valves 402, e.g., opening the valves 402 to select gas inlet 404a.

Data processing apparatus 344 collects response data generated by each gas sensor module 300 of the second subset of gas sensor modules 300 in response to the test gas 406, where the generated response data 118 from each gas sensor of the multiple gas sensors 108 in each respective module 300 of the second subset of gas sensor modules includes a response of the gas sensor to the one or more analytes of the multiple known analytes in the test gas 406. Data processing apparatus 344 can collect response data 118, annotation data 120, and composition data 122 for each gas sensor 108 in response to the test gas, as described with reference to FIG. 1.

The first set of response data and the second set of response data is provided as training data to a machine-learned model (616). A machine-learned model can be trained using the first and second sets of response data 118 to detect the multiple analytes of interest in an unknown test gas for a particular environment/application. Moreover, the machine-learned model can further be trained to determine, based on an application specific set of analytes of interest, a sampling sequence to utilize to test for the application-specific set of analytes of interest including a respective subset of the modules 300 of the multiple modules 300 of an e-nose gas sensing system 340 to detect each of the analytes of interest.

In some implementations, prior to exposing the gas sensors to second test gas 406, the multiple gas sensors 108 are exposed to a purge gas, e.g., nitrogen or compressed clean dry air, for a period of time. The purge gas, e.g., gas source 130a, can be used to assist in shortening a recovery time of the sensors after exposure to the test gas and/or to ensure that no remaining test gas is present within a module 300 or gas manifold 126.

In some implementations, the period of time of exposure of the multiple gas sensors 108 to the purge gas can be an amount of time for each of the sensors to reach a baseline resistivity reading. The period of time of exposure can be selected based on a longest recovery time of all the respective recovery times for the multiple gas sensors 108. The period of time exposure of the multiple gas sensors 108 to the purge gas can be, for example, 1 minute, 5 minutes, 30 seconds, or the like.

The process described with reference to Steps 602-616 can be repeated for multiple test gases each including multiple analytes of interest, where each test gas is representative of the test environment. The multiple test gases can be selected based on a range of compositions and/or environmental conditions, e.g., temperature, over which the response of the gas sensors 108 are measured to generate the training data.

An e-nose gas sensing system 340 including multiple selected modules 300 can be deployed into a test environment to “sniff” for a set of analytes of interest, where the modules 300 are selected based on the set of analytes of interest that are representative of the test environment. In some implementations, training data for an e-nose gas sensing system 340 including a particular set of modules 300 can be generated while the system 340 is deployed in a test environment. The training data can be utilized to re-train and/or refine the machine-learned model that identifies the set of analytes of interest in the unknown test gas in the test environment.

FIG. 7 is a flow diagram of another example process of the e-nose gas sensing system. Test instructions are received by a data processing apparatus and for a test environment to test for multiple analytes of interest representative of the test environment (702). Test instructions, e.g., from a user, can be received by data processing apparatus 344. In some implementations, a user can provide test instructions to the data processing apparatus 344 through user device 414 or through display 410, e.g., touch-screen display, on e-nose gas sensing system 340. In some implementations, test instructions can be automatic or semi-automatic instructions to periodically sample the unknown test gas 501 present in an environment surrounding the e-nose gas sensing system 340.

Test instructions can include a natural language statement from a user, e.g., “measure ripeness of bananas” or “check for presence of pesticides.” Test instructions can define multiple analytes of interest or can define one or more general categories of analytes of interest. The data processing apparatus can receive the test instructions and perform natural language processing to determine a set of analytes of interest to detect in the unknown test gas 501 in the environment 500.

A sampling sequence is determined by the data processing apparatus and using a machine-learned model trained for the test environment (704), where for each analyte of interest, a subset of multi-modal gas sensor modules of multiple multi-modal gas sensor modules is selected that is sensitive to the analyte of interest (706).

The sampling sequence can be based in part on a set of analytes of interest that are defined by the test instructions, where one or more modules 300 of the multiple modules included in the system 340 are identified, e.g., using the machine-learned model, as sensitive to one or more of the set of analytes of interest. The sampling sequence can depend in part, for example, on a response time of the modules 300 to the one or more analytes of interest, a recovery time of the modules 300 to the one or more analytes of interest, etc. For example, if a first module 300 is identified as sensitive to analyte A and B and a second module is identified as sensitive to analyte C, a sampling sequence may be selected to detect the analyte A using the first module, then switch to detect an analyte C using a second module while the gas sensors 108 of the first module 300 recover, and then finally detect analyte B using the first module after the gas sensors 108 of the first module 300 have recovered, e.g., reached a baseline response.

Exposing, according to the sampling sequence, the selected subsets of multi-modal gas sensor modules to an unknown test gas from the test environment (708). As described above with reference to FIGS. 4 and 5, the selected subset of multi-modal gas sensor modules 300 can be selectively exposed to the unknown mixture 501 using the set of valves 402, where opening the selected valves 402 to the selected subset of modules 300 causes the unknown test gas 501 to be exposed to the respective gas sensors 108 of each of the selected subset of modules 300.

Collect, by the data processing apparatus, response data for each of the multiple analytes of interest from the respective selected subsets of multi-modal gas sensor modules according to the sampling sequence (710). The data processing apparatus 344, using a machine-learned model, can then analyze the response data and determine a characteristics, e.g., a concentration level, of the analytes of interest based on the response data collected by the respective subsets of multi-modal gas sensor modules 300.

In some implementations, the characteristics of the analytes of interest determined by the machine-learned model can include higher-level identifiers for the test environment including the unknown test gas 501, e.g., object identifiers, classifications, or the like. For example, the machine-learned model can determine the presence of a particular pesticide and identify, to a user, the particular pesticide by a brand name based on the response data. In another example, the machine-learned model can determine the presence of a ripe banana in the test environment based on the response data, e.g., based on the characteristics of the analytes of interest. The data processing apparatus may provide to a user a natural language answer, e.g., “the banana is ripe.”

In some implementations, response data for an analyte of interest from the selected subset of modules 300 can be a “zero-response” or a response that is below a threshold response. In other words, the system 300 may attempt to sniff for a particular analyte of interest and find that the amount of the particular analyte of interest in the unknown test gas 501 is “zero” or below a threshold amount, e.g., below a detectable range.

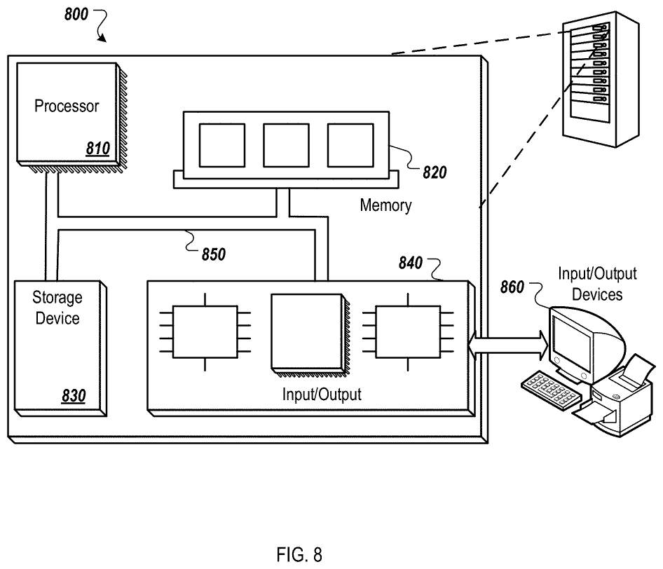

FIG. 8 is a block diagram of an example computer system 800 that can be used to perform operations described above. The system 800 includes a processor 810, a memory 820, a storage device 830, and an input/output device 840. Each of the components 810, 820, 830, and 840 can be interconnected, for example, using a system bus 850. The processor 810 is capable of processing instructions for execution within the system 800. In one implementation, the processor 810 is a single-threaded processor. In another implementation, the processor 810 is a multi-threaded processor. The processor 810 is capable of processing instructions stored in the memory 820 or on the storage device 830.

The memory 820 stores information within the system 800. In one implementation, the memory 820 is a computer-readable medium. In one implementation, the memory 820 is a volatile memory unit. In another implementation, the memory 820 is a non-volatile memory unit.

The storage device 830 is capable of providing mass storage for the system 800. In one implementation, the storage device 830 is a computer-readable medium. In various different implementations, the storage device 830 can include, for example, a hard disk device, an optical disk device, a storage device that is shared over a network by multiple computing devices (for example, a cloud storage device), or some other large capacity storage device.

The input/output device 840 provides input/output operations for the system 800. In one implementation, the input/output device 840 can include one or more network interface devices, for example, an Ethernet card, a serial communication device, for example, a RS-232 port, and/or a wireless interface device, for example, a 802.11 card. In another implementation, the input/output device can include driver devices configured to receive input data and send output data to other input/output devices, for example, keyboard, printer and display devices 860. Other implementations, however, can also be used, such as mobile computing devices, mobile communication devices, set-top box television client devices, etc.

Although an example processing system has been described in FIG. 8, implementations of the subject matter and the functional operations described in this specification can be implemented in other types of digital electronic circuitry, or in computer software, firmware, or hardware, including the structures disclosed in this specification and their structural equivalents, or in combinations of one or more of them.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, that is, one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, for example, a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, for example, an FPGA (field programmable gate array) or an ASIC (application-specific integrated circuit). The apparatus can optionally include, in addition to hardware, code that creates an execution environment for computer programs, for example, code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, for example, an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a Global Positioning System (GPS) receiver, or a portable storage device, for example, a universal serial bus (USB) flash drive, to name just a few.

Computer-readable media suitable for storing computer program instructions and data include all forms of non-volatile memory, media and memory devices, including by way of example semiconductor memory devices, for example, EPROM, EEPROM, and flash memory devices; magnetic disks, for example, internal hard disks or removable disks; magneto-optical disks; and CD-ROM and DVD-ROM disks.

To provide for interaction with a user, embodiments of the subject matter described in this specification can be implemented on a computer having a display device, for example, a CRT (cathode ray tube) or LCD (liquid crystal display) monitor, for displaying information to the user and a keyboard and a pointing device, for example, a mouse or a trackball, by which the user can provide input to the computer. Other kinds of devices can be used to provide for interaction with a user as well; for example, feedback provided to the user can be any form of sensory feedback, for example, visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of messages to a personal device, for example, a smartphone that is running a messaging application and receiving responsive messages from the user in return.

Data processing apparatus for implementing machine learning models can also include, for example, special-purpose hardware accelerator units for processing common and compute-intensive parts of machine learning training or production, that is, inference, workloads.

Machine learning models can be implemented and deployed using a machine learning framework, for example, a TensorFlow framework, a Microsoft Cognitive Toolkit framework, an Apache Singa framework, or an Apache MXNet framework.

Embodiments of the subject matter described in this specification can be implemented in a computing system that includes a back-end component, for example, as a data server, or that includes a middleware component, for example, an application server, or that includes a front-end component, for example, a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, for example, a communication network. Examples of communication networks include a local area network (LAN) and a wide area network (WAN), for example, the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, for example, an HTML page, to a user device, for example, for purposes of displaying data to and receiving user input from a user interacting with the device, which acts as a client. Data generated at the user device, for example, a result of the user interaction, can be received at the server from the device.

While this specification contains many specific implementation details, these should not be construed as limitations on the scope of any features or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method for a multi-modal gas sensing system comprising:
selecting, from a plurality of multi-modal gas sensor modules, a set of multi-modal gas sensor modules, wherein each multi-modal gas sensor module comprises a plurality of gas sensors, and wherein each of the multi-modal gas sensor modules is sensitive to a respective set of analytes;
providing the set of multi-modal gas sensor modules to a receptacle configured to receive the plurality of multi-modal gas sensing modules and comprising a gas manifold in fluidic contact with each of the set of multi-modal gas sensor modules, wherein each of the set of multi-modal gas sensor modules received by the receptacle is in data communication with a data processing apparatus;
configuring, by the data processing apparatus, a sampling sequence for the set of multi-modal gas sensor modules based in part on a plurality of analytes of interest representative of a test environment, the configuring comprising:
determining, by the data processing apparatus, for each analyte of interest of the plurality of analytes of interest, one or more of the set of multi-modal gas sensor modules that is sensitive to the analyte of interest;
selecting, by the data processing apparatus, a first proper subset of the set of multi-modal gas sensor modules to receive a test gas comprising the plurality of analytes of interest based in part on a sensitivity of each multi-modal gas sensor module in the proper subset to the plurality of analytes; and
selecting, by the data processing apparatus, a second proper subset of the set of multi-modal gas sensor modules to receive the test gas comprising the plurality of analytes of interest based in part on a sensitivity of each multi-modal gas sensor module in the proper subset to the plurality of analytes;
providing, to the first proper subset of multi-modal gas sensor modules, the test gas and collecting a first set of response data;
providing, to the second proper subset of multi-modal gas sensor modules and subsequent to the collection of the first set of response data, the test gas and collecting a second set of response data; and
providing, as training data to a machine-learned model, the first set of response data and the second set of response data.

2. The method of claim 1, wherein the first proper subset of multi-modal gas sensor modules is sensitive to a first set of analytes of interest of the plurality of analytes of interest, and the second proper subset of multi-modal gas sensor modules is sensitive to a second, different set of analytes of interest of the plurality of analytes of interest.

3. The method of claim 1, further comprising:
exposing, after providing the test gas to the first proper subset of multi-modal gas sensor modules, the first proper subset of multi-modal gas sensor modules to an inert gas for a period of time.

4. The method of claim 3, wherein the period of time is an amount of time for each of the plurality of sensors in the multi-modal gas sensor modules to reach a baseline resistivity reading.

5. The method of claim 1, wherein the response data from the first proper subset and the second proper subset of multi-modal gas sensor modules comprises a time-dependent response of each of the plurality of gas sensors of each of the multi-modal gas sensor modules in the first proper subset and the second proper subset to the test gas.

6. The method of claim 5, wherein the response data from the first proper subset of multi-modal gas sensor modules is characteristic of a first analyte of interest of the plurality of analytes of interest, and wherein the response data from the second proper subset of multi-modal gas sensor modules is characteristic of a second, different analyte of interest of the plurality of analytes of interest.

7. A multi-modal gas sensing system comprising:
a plurality of multi-modal gas sensor modules, wherein each multi-modal gas sensor modules comprises:
a plurality of gas sensors including a first type of gas sensor and a second type of gas sensor different from the first type of gas sensor, wherein each of the first type of gas sensor and second type of gas sensor is sensitive to a respective set of analytes;
a housing configured to hold the plurality of gas sensors;
a gas inlet coupled to the housing and configured to expose the plurality of gas sensors to a gas introduced from the test environment via the gas inlet; and
a power supply configured to provide power to the plurality of gas sensors;
a receptacle configured to receive the plurality of multi-modal gas sensing modules; and
a data processing apparatus in data communication with the plurality of gas sensing modules and configured to perform the operations comprising:
receiving, by the data processing apparatus and for a test environment, test instructions to test for a plurality of analytes of interest representative of the test environment;
determining, by the data processing apparatus and using a machine-learned model trained for the test environment, a sampling sequence to detect the plurality of analytes of interest, wherein determining the sampling sequence to detect the plurality of analytes of interest comprises:
for each analyte of interest of the plurality of analytes of interest: selecting, a subset of multi-modal gas sensor modules of the plurality of multi-modal gas sensor modules that is sensitive to the analyte of interest; and
collecting, by the data processing apparatus, response data for each of the plurality of analytes of interest from the respective multi-modal gas sensor modules according to the sampling sequence.

8. The system of claim 7, further comprising a gas manifold in fluidic contact with the plurality of multi-modal gas sensing modules and configured to direct the gas to the plurality of multi-modal gas sensing modules.

9. The system of claim 8, wherein the gas manifold is configured to direct the gas to a subset of multi-modal gas sensing modules of the plurality of multi-modal gas sensing modules at a particular time.

10. The system of claim 7, wherein the receptacle comprises circuitry to form i) module-to-module and ii) module-to-platform integration connections between the plurality of multi-modal gas sensing modules and the data processing apparatus.

11. The system of claim 10, further comprising a plurality of circuitry comprising:
sensor interrogation circuitry;
sensor tuning circuitry; and
communication circuitry.

12. The system of claim 7, wherein the plurality of multi-modal gas sensing modules comprise a first multi-modal gas sensing module sensitive to a first set of analytes, and a second multi-modal gas sensing module sensitive to a second, different set of analytes.

13. The system of claim 12, wherein the first multi-modal gas sensing module and the second multi-modal gas sensing module are configured to receive the gas introduced from the test environment via respective gas inlets in parallel.

14. The system of claim 7, wherein the plurality of multi-modal gas sensing modules comprise two or more of a same multi-modal gas sensing module sensitive to a same set of analytes.

15. The system of claim 14, wherein the two or more of the same multi-modal gas sensing module are configured to receive the gas introduced from the test environment via respective gas inlets in series.

16. The system of claim 15, wherein the data processing apparatus collects response data responsive to the gas from the test environment from only a subset of gas sensors of the plurality of gas sensors of one or more respective multi-modal gas sensing modules.
